(19) United States
(12) Patent Application Publication
(10) Pub. No.: US 2012/0097177 A1
(43) Pub. Date: Apr. 26, 2012

(54) Phynlly philters internal nose, ear and mouth filters

(76) Inventor: Paula E. Boothe, Hemet, CA (US)

(21) Appl. No.: 12/925,639

(22) Filed: Oct. 26, 2010

(51) Int. Cl. A62B 23/06; A61F 11/08; A62B 7/10

(52) U.S. Cl. 128/864; 128/206.11; 128/205.29

(57) ABSTRACT

The present invention provides compact nasal, oral and ear inserts with incorporated filters, specially designed to provide a more pleasant and healthy intake of air. Largely fabricated of a soft, pliable fabric such as compressed cotton, each Philter insert is comprised of two cylindrical filters, joined together via a flexible wire bridge. One example of the filtering material is activated charcoal, long known by the medical industry to be a safe, effective filtering agent for extracting liquids and gases from their harmful elements. As an additional consideration, this product is offered in several sizes to accommodate both child (under age twelve would require adult supervision) and adult users. The Philter inserts can be sold individually, or packaged with a dozen of more units per box.

### CLAIM OF PRIORITY

This patent application claims priority under 35 USC 119(e)(1) from U.S. Provisional Patent Application Ser. No. 61/1279,871 filed Oct. 27, 2009, of common inventorship herewith entitled, “PHYNLLY PHILTERS INTERNAL NOSE, EAR AND MOUTH FILTERS.”

### FIELD OF THE INVENTION

The present invention pertains to the filed of air filters for filtering air for human intake.

### BACKGROUND OF THE INVENTION

The prior art has put forth several designs for air filters for use by humans. Among these are:

U.S. Pat. No. 4,996,983 to Floyd E. AmRhein describes an inhaler filtration device with housing supportable by spectacle temple. This reference describes a filter, which attaches to the sides of a pair of eyeglasses.

U.S. Pat. No. 6,543,450 to John T. Flynn describes a survival mask for use in firefighting.

U.S. Pat. No. 7,044,127 to Fernandez DeCastro describes a protective multipurpose mask that covers the oral and nasal areas.

None of these prior art references describe the present invention.

### SUMMARY OF THE INVENTION

It is an object of the present invention to provide improved internal nose, ear and mouth filters.

### BRIEF DESCRIPTION OF THE DRAWINGS

FIG. 1 shows nose filters of the present invention.

FIG. 2 shows ear filters of the present invention.

FIG. 3 shows a cross section showing the layers comprising the filters of the present invention.

FIG. 4 shows a mouth filter of the present invention.

FIG. 5 shows a back view of the mouth filter of the present invention.

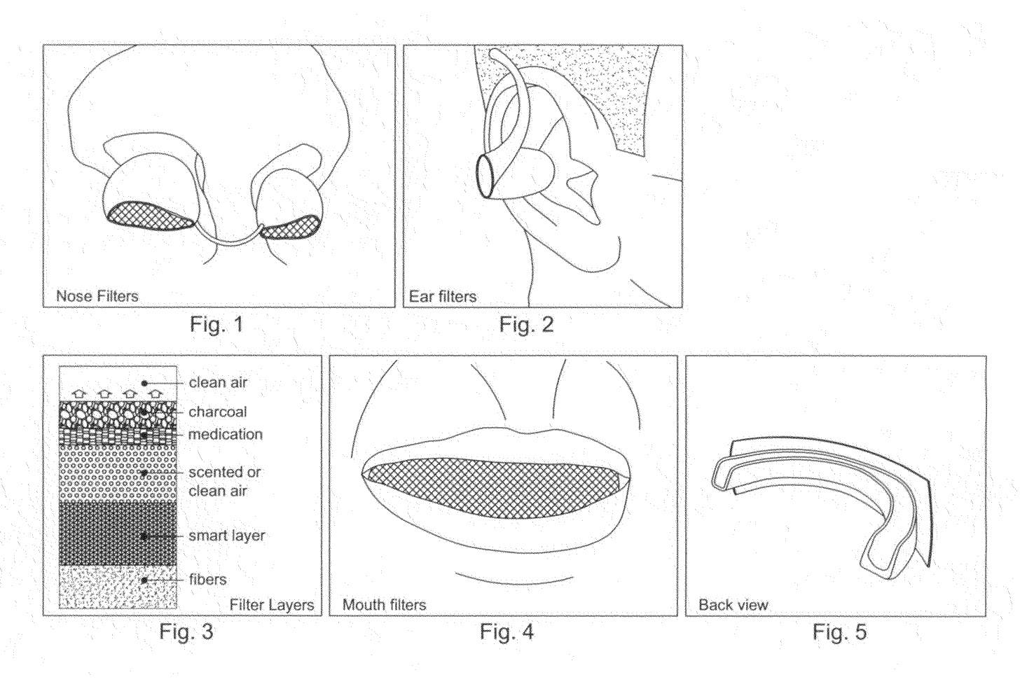

### DETAILED DESCRIPTION OF THE INVENTION

Allergies are among the most common of medical disorders and are abnormal reactions of the immune system, which occur in response to otherwise harmless substances. It is estimated that 60 million Americans, or more than one in every five people, suffer from some form of allergy, with similar proportions throughout much of the rest of the world. In fact, allergy is the single largest reason for school absence and is a major source of lost productivity in the workplace. An allergy is a type of immune reaction. Normally, the immune system responds to foreign bodies, like pollen or bacteria, by producing specific proteins called antibodies that are capable of binding to identifying molecules (antigens) on the foreign body. This reaction between antibody and antigen sets off a series of reactions designed to protect the body from infection. Sometimes, this same series of reactions is triggered by harmless, everyday substances. This is the condition known as allergy, and the offending substance is called an allergen. Allergens enter the body through four main routes: the airways, the skin, the gastrointestinal tract, and the circulatory system. The most common of allergens, airborne allergens cause the sneezing, runny nose, and itchy, bloodshot eyes of hay fever (allergic rhinitis). Whether dust, mold, pollen, or pet dander, airborne allergens can also affect the lining of the lungs, causing asthma, or the conjunctiva of the eyes, causing conjunctivitis (pink eye).

To combat these nuisances in the home and avoid allergic reactions, many consumers employ equipment such as dehumidifiers to dry the air, thus preventing a suitable environment for allergens to breed, as well as air purifiers that capture the airborne particles through a filtering system. While these devices are quite effective in reducing allergens in the home, they tend to be quite expensive, and are simply not practical for most allergy sufferers. In addition, dehumidifiers and air purifiers are not portable, so those who depend upon them find themselves at the mercy of allergens when going to work or attending to outside tasks and errands. Susceptible to reactions from airborne allergens swirling outside and in public buildings, allergy sufferers face daunting challenges whenever they leave the safety of home.

Recognizing the need for an affordable and portable means of combating allergens, the present invention hereinafter referred to as Phynlly Philters, was developed. Simply stated, this product is a line of compact nasal, oral and ear inserts with incorporated filters, specially designed to provide a more pleasant and healthy intake of air. Largely fabricated of a soft, pliable fabric such as compressed cotton, each Philter insert is comprised of two cylindrical filters, joined together via a flexible wire bridge. One example of the filtering material is activated charcoal, long known by the medical industry to be a safe, effective filtering agent for extracting liquids and gases from their harmful elements. As an additional consideration, this product is offered in several sizes to accommodate both child (under age twelve would require adult supervision) and adult users. The Philter inserts can be sold individually, or packaged with a dozen of more units per box.

Use of Phynlly Philters is very simple and straightforward. First, the user selects inserts based on personal preference of size. By way of example, a mother may choose a small size package for her daughter who is allergic to dust. Similarly, a construction worker may need a larger unit for use when out on the job. Removing a Philter unit from its packaging, the user simply inserts a filter into each nostril, pushing upward until the wire bridge rests flush against the columella of the nose, or the area between the nostrils. While inside the nose, the Phynlly Philters insert works to capture airborne allergens within its filters, before they enter the airways.

In addition, as the user inhales, the filter absorbs pathogens and odors, attaching to them by chemical attraction. The surface area of activated charcoal gives it countless bonding sites, and when certain chemicals pass next to the carbon surface, they attach to the surface and are trapped. Thus, the filters cover up any external odors that may be circulating. After use, the Phynlly Philters insert can be easily removed, and simply discarded in the closest trash receptacle.

Phynlly Philters afford consumers a number of significant benefits and advantages. Foremost, this clever invention provides a simple, affordable means for allergy sufferers to combat airborne allergens at any time of day, wherever they may go. A compact filtering system that can be inserted directly into the nose, Phynlly Philters offers a portable, low cost solution for blocking out pollen, dust, mold, or pet dander in the home, in the office, outdoors, or in other public buildings. In addition to fighting allergens, this versatile product's absorbency capabilities also effectively resists foul odors. In this manner, non-allergic consumers can purchase Phynlly Philters to keep on hand whenever encountering a foul stench, such as driving through an area that reeks from pollution or odors emanating from crowds on public transportation. Extremely easy to use, a Phynlly Philter insert can be applied and removed in just a matter of seconds. Safe for all users and offered in various sizes, this practical invention is ideal for children as well as adults. This technology can be expanded to filters for the ears and the mouth.

An innovative invention, Phynlly Philters offers allergy sufferers a more practical tool in the battle against airborne allergens. Lightweight and comfortable, this handy invention provides a portable filtering system whenever one is needed. Affordably priced, Phynlly Philters will be well received by the general consumer populace, particularly those with allergies, a very sizable market potential.

Although this invention has been described with respect to specific embodiments, it is not intended to be limited thereto and various modifications which will become apparent to the person of ordinary skill in the art are intended to fall within the spirit and scope of the invention as described herein taken in conjunction with the accompanying drawings and the appended claims.

1. A filter for inserting into the nose, mouth or ear of a human, comprising:
compact filters comprising an insert wherein the insert comprises a soft, pliable fabric, and is comprised of two cylindrical filters, joined together by a flexible wire bridge.

2. The filter of claim 1 wherein the filter comprises activated charcoal.

3. The filter of claim 1 wherein the soft, pliable fabric comprises compressed cotton.

4. The filter of claim 1 sized to accommodate the head and face of a child.

5. The filter of claim 1 sized to accommodate the head and face of an adult.
